(19) United States
(12) Patent Application Publication
(10) Pub. No.: US 2021/0330413 A1
(43) Pub. Date: Oct. 28, 2021

(54) MEDICAL ISOLATION DEVICES AND METHODS

(71) Applicant: Freshwater Holdings LLC, Traverse City, MI (US)

(72) Inventors: Anastacia Feeley, Traverse City, MI (US); Kristin Bauman, Traverse City, MI (US); Kristen Kurjan, Traverse City, MI (US); Aaron Kurjan, Traverse City, MI (US)

(21) Appl. No.: 17/237,838

(22) Filed: Apr. 22, 2021

Related U.S. Application Data
(60) Provisional application No. 63/014,097, filed on Apr. 22, 2020.

(51) Int. Cl. A61B 46/23

(52) U.S. Cl. CPC A61B 46/23; A61B 2217/005; A61B 2562/0247

(57) ABSTRACT

A medical isolation device for use by a medical professional to safely create a barrier between the medical professional and an ill patient in need of treatment, such as intubation. The medical isolation device has a body, a drape, a porthole, and a porthole cover. The medical isolation device may also have ports, such as suction and oxygen ports. The body and the drape form the medical isolation device that may be placed over the head and neck of a patient. The body extends between a base edge and a front side edge, formed among a first side edge, a second side edge, a top side edge, and a back side edge.

### CROSS-REFERENCE TO PRIOR FILED APPLICATIONS

This application incorporates by reference and claims the benefit of priority to U.S. Provisional Application No. 63/014,097 filed on Apr. 22, 2020.

### BACKGROUND OF THE INVENTION

The present subject matter relates generally to a medical isolation device that isolates the head of a patient and the surrounding air from the ambient environment within a room. More specifically, the present subject matter provides a medical isolation device including a draped body that is placed over the patient's head while lying on a table.

The COVID-19 pandemic has flooded emergency rooms and intensive care units across the world with patients suffering from the virus, and this is simply one example infectious disease. As infectious diseases are transmitted between people in close proximity through physical contact and/or airborne droplets, treating patients infected with COVID-19 is proving to be exceptionally difficult. Respiratory distress is a common complication often requiring intubation. In a typical emergency environment, such as a patient being brought into an emergency room via ambulance with the patient exhibiting severe and acute symptoms of COVID-19, medical professionals are left with few options to protect themselves and support staff. The medical staff is exposed to contaminated air or contagions expelled from the patient into the room, and are at risk of direct transmission via contact with contagions and/or airborne droplets of known and unknown illnesses. At the same time, patients are exposed to potentially contaminated air from other patients or carriers.

In addition to emergency rooms, a multitude of environments pose serious risks to medical professionals and those taking care of infected persons: the operating room for induction and emergence of anesthesia and throughout surgical procedures, intensive care units (ICUs), endoscopy suites, radiology suites, magnetic resonance imaging (MRI) procedures, ear, nose, and throat (ENT) procedures, emergency medical technician (EMT) and paramedic vehicles, dental offices, patient transportation, hospice, and any other situation or location where medical care is provided. Further, medical and non-medical persons are even more limited in protection when caring for loved ones or others at home during end of life care.

Medical professionals wear standard personal protective equipment (PPE) while working in these environments to prevent the transmission of the infection. PPE, however, is not 100% effective in blocking transmission. Further, medical professionals also have difficulty donning and doffing personal protective equipment. For example, the excess time spent for each person in an emergent situation to don personal protective equipment and address time sensitive ailments of a patient may cause a failure to timely treat an acute issue or force medical professionals to make choices about their own safety.

Accordingly, a need exists for a medical isolation device to isolate an infected patient and the patient's airflow from the surrounding environment, and/or isolate a non-infected patient and the patient's airflow from surrounding contaminated air during a high volume turnover in emergency rooms during a pandemic. There exists a need for a medical isolation device that the medical professionals and patients have discreet respiratory environments from each other, as described herein.

### BRIEF SUMMARY OF THE INVENTION

To meet the needs described above, the present application provides a medical isolation device to isolate a patient and the patient's airflow, such that the medical professionals and patients have discreet respiratory and physical environments from each other.

In an embodiment, the medical isolation device includes a body and a drape extending from the body to create an enclosed space. The body includes at least one porthole and a porthole cover within the porthole to enable the medical personnel to access the patent within the body. An oxygen port and a suction port on the body provide passageways through the body to enable air circulation for the patient without exposing medical personnel to the infected air.

The body includes a first side portion, a second side portion, a top portion, and a back portion. A front side edge is formed along a first edge of the first side portion, a first edge of the second side portion, and an edge of the top side portion. A base edge is formed along a second edge of the first side portion, a second edge of the second side portion, and an edge of the back side portion. The drape is attached to the front side edge of the body and extends at least the full height and width of the front side opening.

One or more portholes provide a throughway in the body through which medical devices such as but not limited to intubation equipment, hands, arms, and other items can be placed. The portholes include a porthole cover that fits securely into the porthole and limits the exchange of air in and out of the body of the medical isolation device. The porthole cover includes an outer edge that mates with a porthole edge of the porthole and an inner edge spaced from the outer edge defining the throughway. The porthole cover is removable from the porthole for immediate access to the patient in an emergency situation as well as for cleaning purposes.

The porthole cover is biased into a folded position such that the inner edge comes together to close the throughway. Upon the application of force to the porthole cover, the cover unfolds such that the inner edge is drawn apart to open the throughway. The porthole cover is comprised of a material that is flexible in order to unfold upon the application of force but sufficiently rigid to be biased into a folded position and withstand negative pressure within the medical isolation device. One example material is silicone, although other suitable materials may be used.

In some embodiments, the front side edge of the body is flanged. The drape may include a plurality of pegs along an attachment edge, and a flange on the front side edge of the body may include a plurality of holes for mating with the plurality of pegs of the drape. The attachment edge may attach to the front side edge of the body through a hook and loop attachment. In yet another embodiment, the drape has a grooved attachment edge for receiving the flanged front side edge of the body.

In another embodiment, the edge of the porthole is flanged. In yet another embodiment, the outer edge of the porthole cover is grooved for receiving the flanged edge of the porthole.

During use, the combination of the body and the drape, and the ports creates an enclosure that is capable of withstanding negative pressure.

An object of the medical isolation device is isolate air expelled by an infected patient and the ambient environment of the medical professionals treating the patient, or to isolate the air flow of a healthy or immunocompromised patient and an ambient environment at risk of carrying contagions.

A further object of the medical isolation device is to allow access to the patient for procedures, such as intubation, while isolating the respective ambient environments of the patient and medical professionals.

An advantage of the medical isolation device is that medical professionals are not exposed to physical and/or airborne pathogens, such as COVID-19, while treating patients.

A further advantage is the ability to easily sterilize and autoclave the medical isolation device for constant reuse. During a pandemic, the ability to sterilize and autoclave for easy reuse is valuable as supply chain shortages can be anticipated.

Another advantage of the medical isolation device is that patients have improved portability throughout a care facility, such as a hospital, while preventing all encountered persons in the vicinity from breathing any air expelled by the ill patient. Similarly, the medical isolation device improves the containment of pathogens as its use in enclosed spaces such as elevators, hallways, or intermediate rooms in the healthcare facility provides a cleaner environment for all personnel, patients, and visitors.

Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one implementation in accord with the present concept, by way of example only, not by way of limitations. In the figures, like reference numerals refer to the same or similar elements.

FIG. 1 is a perspective view of a medical isolation device of the present application.

FIG. 2 is a front elevational view of the medical isolation device of FIG. 1.

FIG. 3 is a bottom plan view of the medical isolation device of FIG. 1.

FIG. 4 is a plan view of the medical isolation device of FIG. 1.

FIG. 5 is a right side view of the medical isolation device of FIG. 1.

FIG. 6 is a left side view of the medical isolation device of FIG. 1.

FIG. 7 is a front perspective view of a porthole cover next to a porthole of the medical isolation device of FIG. 1.

FIG. 8 is a back perspective view of the porthole cover on the medical isolation device of FIG. 1 with a medical instrument extending therethrough.

FIG. 9 is a front perspective view of a porthole cover used with the medical isolation device of FIG. 1.

FIG. 10 is a back perspective view of the porthole cover of FIG. 9.

FIG. 11 is an enlarged, cross-sectional view of a first embodiment of the drape attached to the front side edge of the body of the medical isolation device of FIG. 1.

FIGS. 12A and 12B are an enlarged, cross-sectional view and an exploded, perspective view of a second embodiment of the drape attached to the front side edge of the body of the medical isolation device of FIG. 1.

FIG. 13 is a perspective view from above of the medical isolation device of FIG. 1 during use.

FIG. 14 is a front elevational view of the medical isolation device of FIG. 1 during use.

FIG. 15 is a top perspective view of a further embodiment of the medical isolation device of the present application shown in the operative position.

FIG. 16 is a bottom perspective view of the medical isolation device of FIG. 15.

FIG. 17 is a top perspective view of the medical isolation device of FIG. 14 shown in the collapsed position.

### DETAILED DESCRIPTION OF THE DRAWINGS

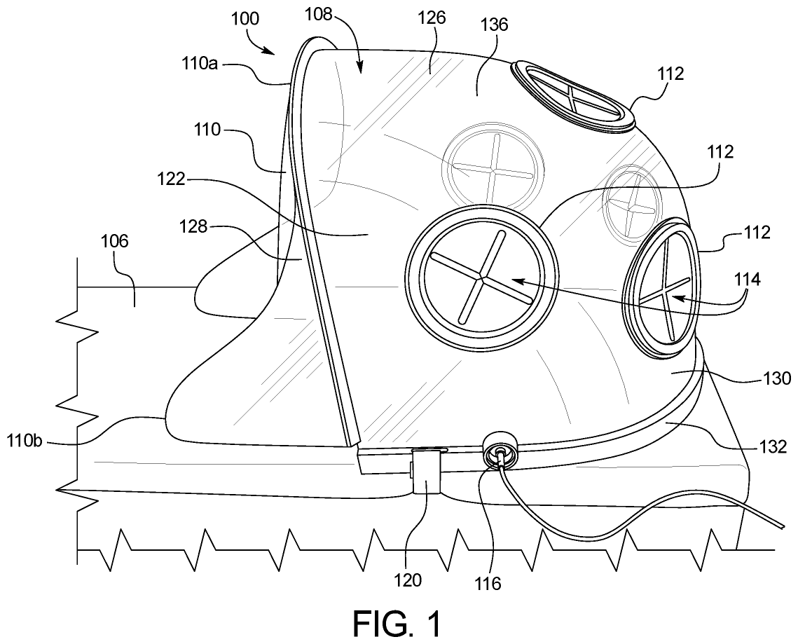

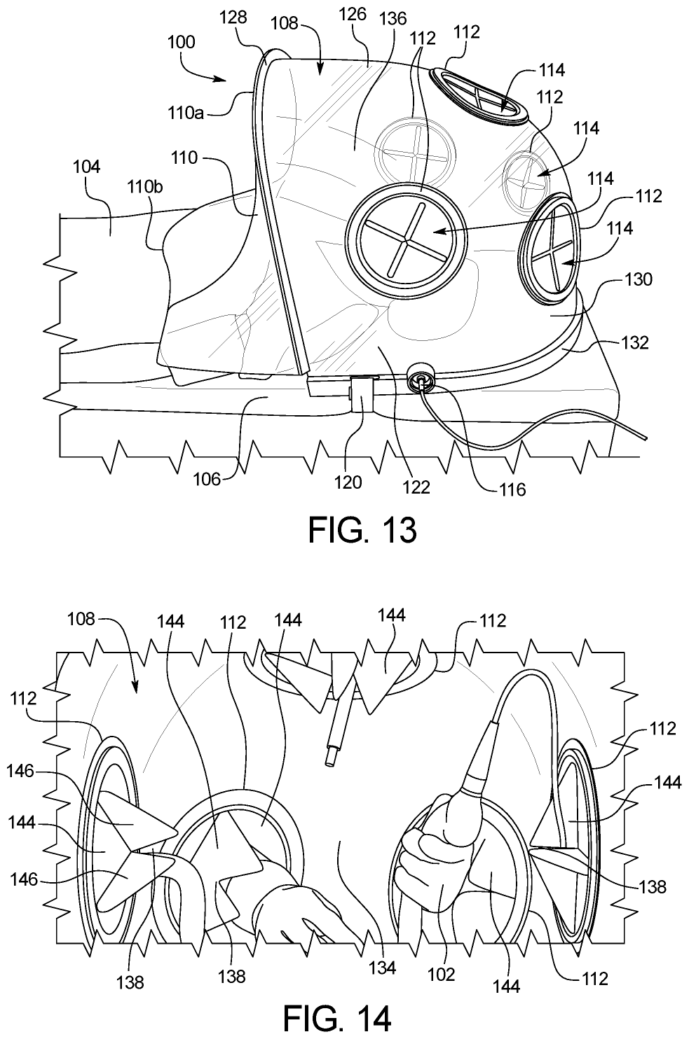

FIGS. 1-14 illustrate an example embodiment of a medical isolation device 100 that is used by medical professionals 102 to cover and enclose the head portion of an infected patient 104 lying on a flat surface 106, such as a medical gurney or bed, as shown in FIGS. 13 and 14.

As shown in FIGS. 1-12, the medical isolation device 100 includes at least a body 108 and a drape 110 as well as passageways through which air, medical devices, and medical personnel can access the patient 104. More specifically, the body 108 includes at least one porthole 112 with a porthole cover 114 that allows medical instruments and tubing as well as the hands and arms of the medical personnel to access the patient 104 through the device 100, seen best in FIG. 14. The medical isolation device 100 also includes an oxygen port 116 and a suction port 118 that enables controlled airflow through the device 100 for the patient 104.

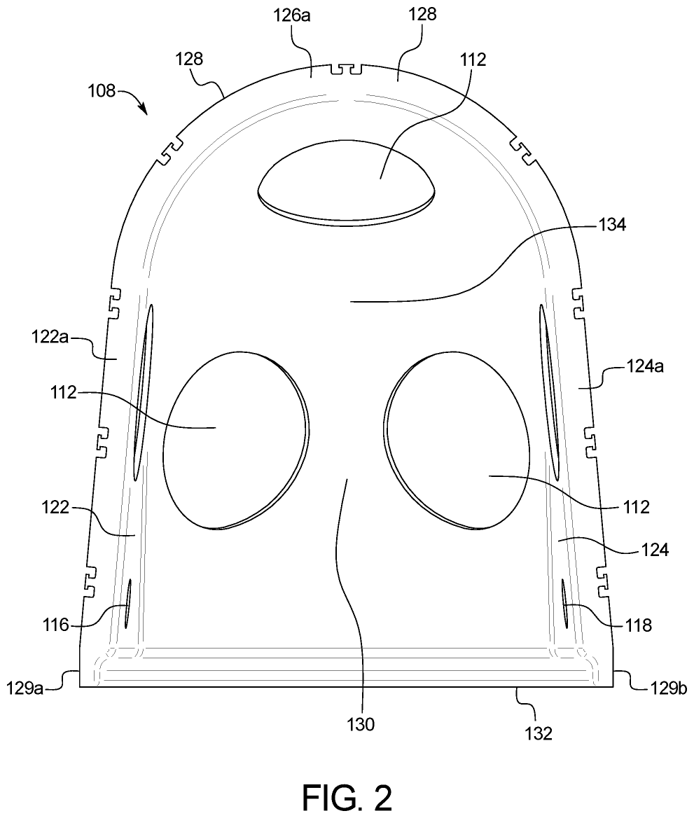

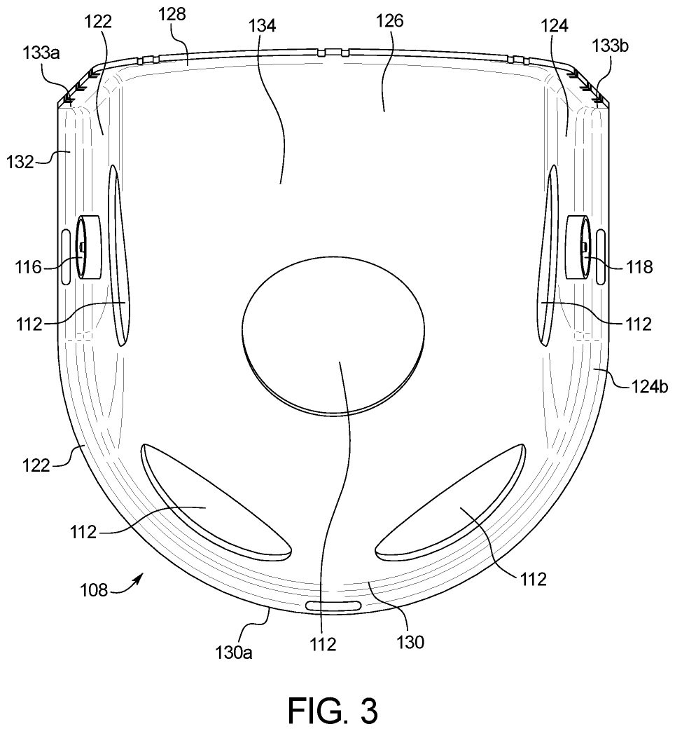

Referring to FIG. 1, the body 108 includes a first side portion 122 and a second side portion 124 opposite of the first side portion 122. A top side portion 126 connects upper edges of the first 122 and second side portions 124, and a front side edge 128 (see FIG. 2) is formed along a first edge 122a of the first side portion 122, an edge 126a of the top side portion 126, and a first edge 124a of the second side portion 124. A back side portion 130 connects the first 122 and second side portions 124, and a base edge 132 (see FIG. 3) is formed along a second edge 122b of the first side portion 122, an edge 130a of the back side portion 130, and a second edge 124b of the second side portion 124.

The body 108 of the medical isolation device 100 illustrated in FIGS. 2-6 has a dome shape extending between the base edge 132 and the front side edge 128, each of the base edge 132 and the front side edge 128 having a U-shape. FIG. 2 illustrates a front side edge 128 having an arcuate shape extending between first and second front edge endpoints 129a, 129b. FIG. 3 illustrates a base edge 132 having an arcuate shape extending between first and second base edge endpoints 133a, 133b. In the illustrated embodiment, the first and second base edge endpoints 133a, 133b and the first and second front side edge endpoints 129a, 129b, respectively, coincide. In other embodiments, the body 108 may have a rectangular shape, a cubical shape, or any other shape providing a sufficient volume within for positioning over the head portion of a patient 104.

The body 108 allows for a patient 104 to be placed within the medical isolation device 100 from at least their shoulders to the top of their head (see FIG. 13). The body 108 may come in a variety of sizes in order for medical professionals 102 to safely care for patients 104 of all ages, shapes, and sizes. In an embodiment, the base edge 132 of the body 108 includes an outer shoulder with one or more openings or slits for receiving a strap 120 that secures the medical isolation device 100 to the flat surface 106. In one example embodiment, the body 108 includes first and second slits on the base edge 132 adjacent to the first side portion 122 and the second side portion 124, respectively. The strap 120 extends through the first slit, along the side of the operating table 106 adjacent to the first side portion 122 of the body 108, under the operating table 106 to the other side of the operating table 106 adjacent the second side portion 124 of the body, and up the other side of the operating table 106 and through the second slit on the base edge 132 adjacent to the second side portion 124 of the body 108. A further strap may extend from a midpoint of the initial strap to a third slit in the base edge 132 adjacent the back side portion 130 of the body 108. In another embodiment, first and second straps 120 extend through first and second slits, respectively, adjacent to the first side portion 122 and the second side portion 124, respectively, and wrap around bars extending along the sides of the operating table or surface. Ends of the strap 120 include a hook and loop attachment or other securement mechanism that enable the ends of the strap 120 to connect to a main portion of the strap, enabling the medical professional or use to easily tighten the device 100 onto the operating table 106. Numerous known substitutes may be used to secure the medical isolation device 100, as will be recognized by those skilled in the art.

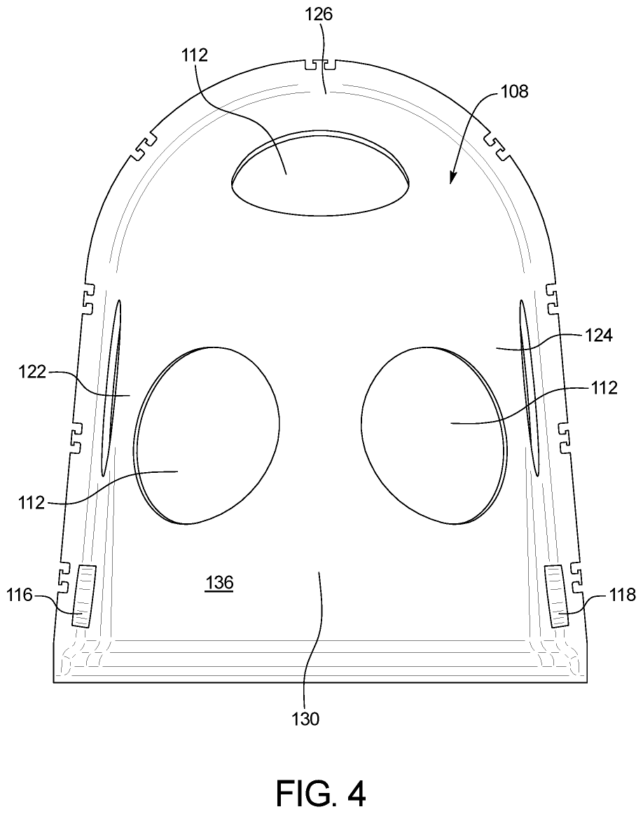

The body 108 includes an inner surface 134 (FIGS. 2 and 3) and an outer surface 136 (FIG. 4) along with passageways for air and instruments. At least one porthole 112 in the body 108 provides access between the outer surface 136 and the inner surface 134 of the body 108. In the medical isolation device 100 illustrated herein, the body 108 includes five portholes 112 for providing access to the interior of the body 108.

Referring to FIG. 7, a porthole cover 114 is positioned within each porthole 112 of the body 108 during use. The porthole cover 114 provides a throughway 138 that is biased into a closed position seen in FIG. 7 and moves into an open position when force is applied as shown in FIG. 8. The porthole cover 114 therefore allows access into the interior of the body 108 while restricting the exchange of air in and out of the medical isolation device 100. The porthole cover 114 is removable from the porthole 112 for immediate access to the patient 104 in an emergency situation as well as for cleaning purposes.

The porthole cover 114 includes an outer edge 140 that mates with a porthole edge 113 of the porthole 112 in the body 108, as shown in FIG. 7. In the illustrated embodiment, the outer edge 140 of the porthole cover 114 includes a U-shaped groove that mates with a porthole edge 113 of the porthole 112. As shown in FIG. 8, an inner edge 142 within the porthole cover 114 spaced from the outer edge 140 defines a throughway 138 in the porthole cover 114, providing passage of air, instruments, and other objects between the inner surface 134 and outer surface 136 of the body 108.

When no force is applied to the porthole cover 114, the cover 114 is biased toward a folded position such that the inner edge 142 comes together to close the throughway 138 as shown in FIG. 7. When the cover is in the folded position, the throughway 138 is closed and air is prevented from moving from the inner surface 134 and outer surface 136 of the body 108. When force is applied to the porthole cover 114, the cover unfolds and the inner edge 142 is drawn apart to open the throughway 138 as shown in FIG. 8. During use, when a medical professional 102 applies force to pass an arm or medical device from outside of the body 108 to the interior of the body 108, the inner edge 142 of the porthole cover 114 is drawn apart to allow access, but remains biased toward the folded position to minimize the exchange of air.

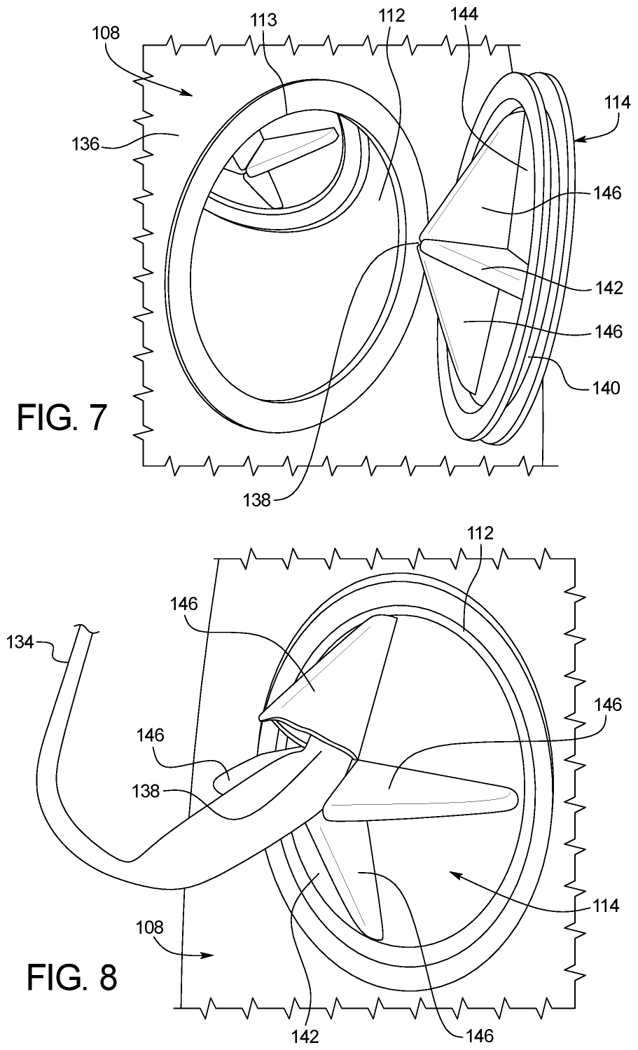

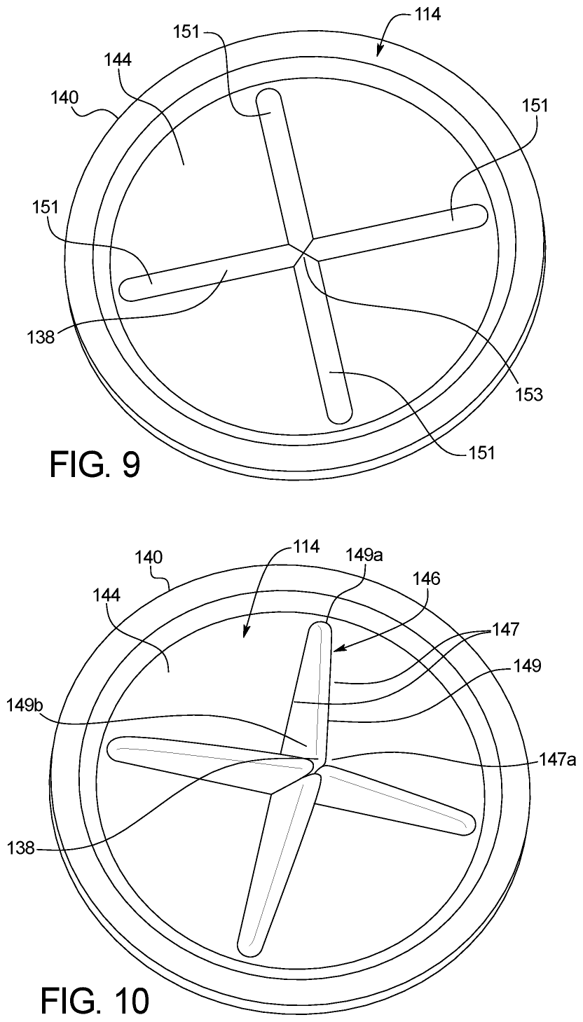

As shown in FIGS. 9 and 10, the porthole cover 114 includes planar portions 144 and folded portions 146. Each folded portion 146 extends from a back side of the porthole cover 114 as seen in FIG. 10, and includes triangularly shaped opposing fold sides 147 that connect along a bridge 149. At a first end 149a of the bridge 149, the opposing fold sides connect to form a corner 151. Each fold side 147 includes a fold side edge 147a that extends between the planar portion 144 of the porthole cover 114 and a second end 149b of the bridge 149. The folds of the plurality of folds are formed within the porthole cover 114 so that the fold side edges connect to form the inner edge 142 of the porthole cover 114.

On a front side of the porthole cover 114, the planar portions 144 of the cover are separated by voids 151 formed by the folded portions 146. More specifically, the fold sides 147 of each folded portion 146 extend away from the planar portions 144, forming narrow cavities 151 between adjacent fold sides 147. During use, the object moving through the porthole cover 114 first contacts the central point 153 of the porthole cover 114. The porthole cover 114 is reversible in that it can also be positioned within the porthole 112 such that the folded portion 146 extends outwardly into the ambient environment instead of inwardly toward the interior of the body 108, as shown in the illustrated embodiment. For example, medical staff may position the cover 114 with the folded portion 146 outside of the body 108 if the respective porthole 112 is used only for moving medical instruments or tubing out of the body 108.

When the porthole cover 114 is in the folded position without force being applied thereto as seen in FIG. 10, each fold 146 may extend from the planar portion 144 of the porthole cover 114 at an angle of about 90 degrees. In other embodiments, the fold sides 147 may extend from the planar surface 144 between about 60 degrees and 90 degrees, coming together to form the fold 146 and maintain a closed throughway 138 along the inner edge 142. The size and shape of the folds 146 may be modified as desired and as needed, depending on material utilized. In another embodiment, the inner edge 142 in the folded position is produced on an angled or twisted orientation to each other for minimal residual opening of the inner edge 142 of the porthole 112.

The size, shape, number, and location of the portholes 112 may vary as desired. The porthole(s) 112 may vary in size according to the instruments or hands and arms of the medical personnel needing to access the patient 104 positioned within the medical isolation device 100. In the illustrated embodiment, the portholes 112 and porthole covers 114 are circular. The shape of the porthole 112 and corresponding porthole cover 114 may be square, rectangular, semicircular, or any other shape as desired. Numerous known sizes and shapes may be used, as will be recognized by those skilled in the art.

The body 108 illustrated in FIGS. 1-12 includes five portholes 112. A first porthole 112 is located directly above the face of the patient 104 in the top side portion 126 of the body 108. Second and third portholes 112 are located within the first 122 and second side portions 124, respectively. Fourth and fifth portholes 112 are located next to one another in the back side portion 130. In other embodiments, additional or fewer portholes 112 and porthole covers 114 may be used.

In a preferred embodiment, the porthole cover 114 is made of a sufficiently flexible material that easily deforms so as to fit within the porthole edge of the porthole 112. The material is also sufficiently rigid so as to maintain its shape and resist pressure when applied thereto. The porthole cover 114 may be made of silicone, polymeric materials, or other suitable materials. Numerous known substitutes may be used, as will be recognized by those skilled in the art.

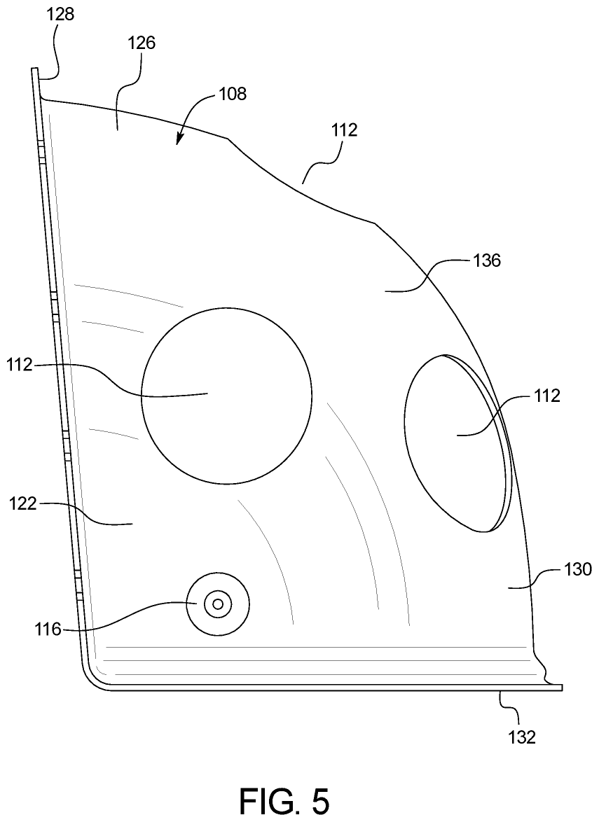

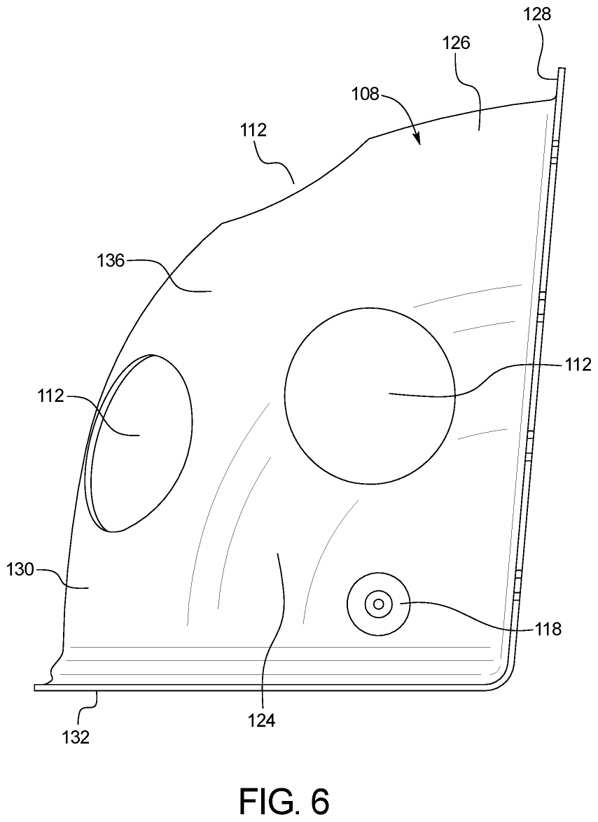

Referring to FIGS. 5 and 6, the oxygen port 116 and suction port 118 are adjacent to the base edge 132 of the body 108. In the illustrated embodiment, the oxygen port 116 is formed within an oxygen connector, and the oxygen connector is positioned within an opening within the body 108. The oxygen port 116 is sized to receive tubing connected to an oxygen source. During use, the oxygen port 116 may receive oxygen from the oxygen source at a flow rate of 6-8 L/min. or other flow rate as needed to attain the desired pressure within the device 100. The suction port 118 is formed within a suction connector that is positioned within an opening in the body 108 near the base edge 132. The suction port 118 is sized to receive tubing connected to waste anesthesia gas system or other suction device. In a further embodiment, suction port 118 can contain a viral filter attachment to use with a transport suction canister or other suction device without filtration when waste anesthesia gas is unavailable.

The oxygen and suction ports 116, 118 may be either pressed into place using a press-fit, twisted into place using a threaded connection, or permanently attached to the body 108. In one embodiment, the oxygen and suction ports 116, 118 are removable and interchangeable on the body 108. The oxygen 116 and suction ports 118 and/or connectors are made of disposable plastic that is standard within the medical industry or a plastic material that may be autoclaved, sterilized, and reused. Numerous known materials may be used, as will be recognized by those skilled in the art. The oxygen and suction ports 116, 118 may be color coded or otherwise labeled for easy identification. Additional accessories, such as pressure indicators, a negative-pressure indicator, a negative pressure device, etc., may also be used with the device 100.

The oxygen port 116 and suction port 118 allow for the creation of negative air pressure within the medical isolation device 100. The pressure differential between the oxygen flow rate and the suction flow rate creates the negative pressure inside the medical isolation device 100, with the suction flow rate exceeding the oxygen flow rate. The oxygen port 116 and suction port 118 can vary in size and function depending on the need of the medical professionals 102 at the time of treatment. The medical isolation device 100 does not require an airtight seal along its edges, but is sufficiently enclosed and comprised of rigid materials to withstand a negative pressure environment within the device 100.

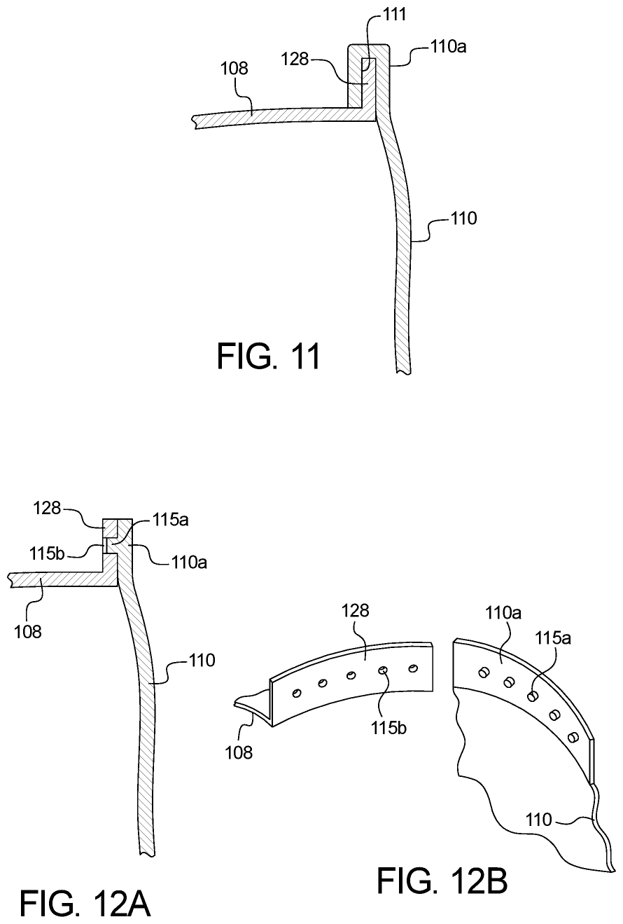

Referring back to FIG. 1, the drape 110 is attached to the body 108 to provide a cover over the front side edge 128 of the body 108. In the embodiment illustrated in FIG. 11, the drape 110 includes an attachment edge 110a that is secured to the front side edge 128 of the body 108. The attachment edge 110a includes a U-shaped groove 111 that mates with the front side edge 128. In another embodiment illustrated in FIGS. 12A and 12B, the attachment edge 110a includes a plurality of pegs 115a that mate with a plurality of openings 115b on the front side edge 128 of the body 108. In other embodiments, the attachment edge 110a may attach to the front side edge 128 via an adhesive or other suitable product or mechanism. In other embodiments, the drape 110 may attach to the top side portion 126 or another element of the body 108. The drape 110 also includes a free edge 110b that rests atop the flat surface 106 and/or the patient's 104 body.

In some embodiments, the drape 110 extends longer than the height of the body 108 such that the drape 110 drapes over the chest, neck, or body of the patient 104 to limit the amount of air that can flow between the inside surface and outside surface of the medical isolation device 100. In an embodiment, the drape 110 is of uniform width at the distal end. In yet another embodiment, the distal end of the drape 110 tapers around the body of the patient 104. In a further embodiment, the drape 110 is supplied in a size matching the size of the body 108 so as to safely cover patients 104 of all ages, shapes, and sizes.

In a preferred embodiment, the body 108 is self-supporting and made of clear polycarbonate or tempered glass. The materials are selected for the transparency so both the patient and the medical personnel have unobstructed views through the device 100. The materials are also resilient to fading and scratching, and allow for easy nesting, stacking, and storage. The materials are also tolerant of repeated autoclaving and disinfecting. The body 108 can be formed from any structural material appropriate for enclosing the internal elements. Numerous known substitutes may be used, as will be recognized by those skilled in the art. The body 108 is reusable and capable of being sanitized sufficiently between uses via methods such as fogging or autoclaving. The drape 110 is reusable and capable of being sanitized sufficiently between uses via methods such as fogging or autoclaving.

The porthole cover 114 is reusable and capable of being sufficiently sanitized between uses via methods, such as fogging or autoclaving. In another embodiment, the medical isolation device 100, including the porthole cover 114, is completely disposable as medical waste, recycling, or any suitable method of discard. Numerous known materials may be used, as will be recognized by those skilled in the art. In a preferred embodiment, the porthole cover 114 is transparent or as close to transparent as the porthole cover's 114 structural material allows.

The drape 110 may be made of a polymeric material that is thin and flexible and can be formed from any polymeric or structural material appropriate for the functions as described herein. Numerous known substitutes may be used, as will be recognized by those skilled in the art. In a preferred embodiment, the drape 110 is transparent or as close to transparent as the drape 110 structural material allows.

In another embodiment, the medical isolation device 100, including the body 108, the drape 110, and the porthole cover(s) 114, is completely disposable as medical waste, recycling, or any suitable method of discard. Numerous known materials may be used, as will be recognized by those skilled in the art. In a preferred embodiment, the body 108 is transparent or as close to transparent as the body's 108 structural material allows.

During use, the user or medical professional 102 positions the medical isolation device 100 atop the flat surface 106 such that the head of the patient 104 is positioned within the body 108 of the medical isolation device 100 as shown in FIG. 13. The user then attaches the drape 110 to the side edge of the body 108 such that a free edge of the drape 110 rests atop a body of the patient 104. The user connects suction tubing to the suction port 118 on the medical isolation device 100 that is connected to a suction device. The user also connects oxygen tubing to the oxygen port 116 on the medical isolation device 100 that is connected to an oxygen source. The use then operates the suction device and the oxygen source to create airflow through the medical isolation device 100 and maintain a negative pressure. With the medical isolation device 100 in place, the user inserts medical devices, instruments, and other components through the porthole covers 114 as needed, as shown in FIG. 14.

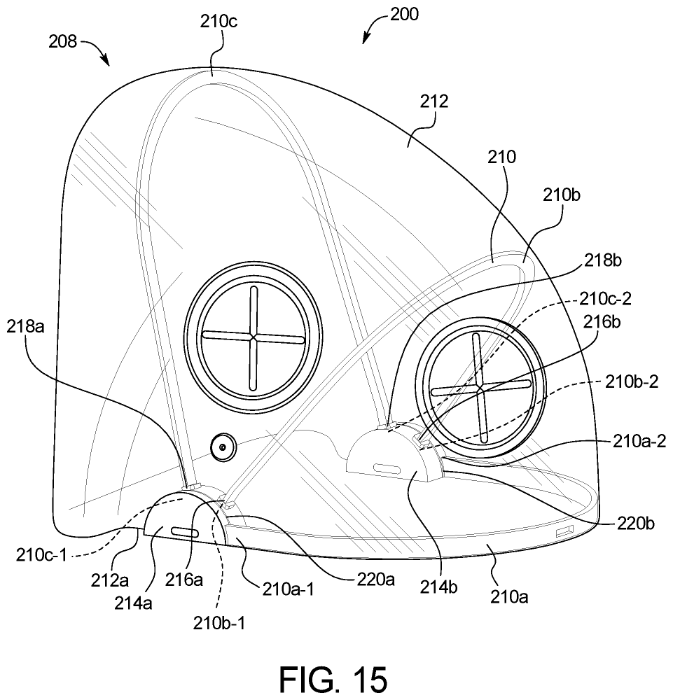

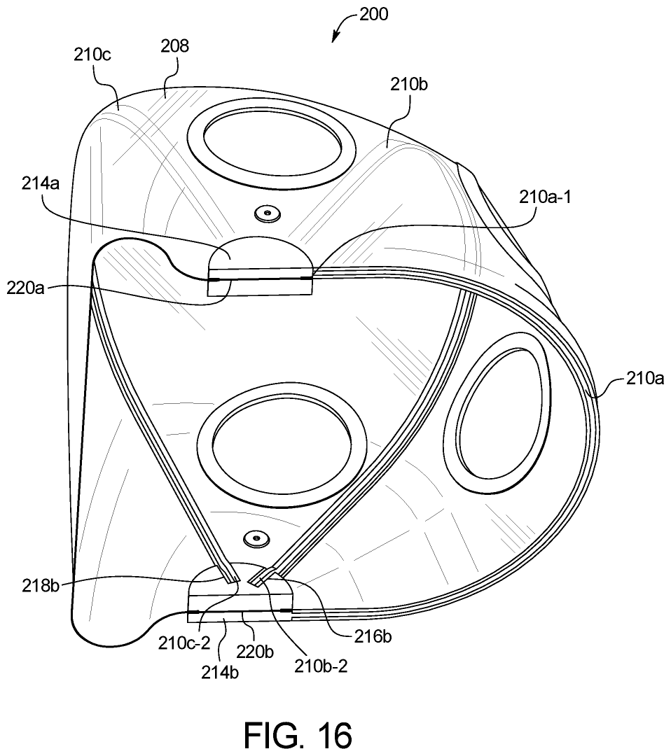

In a further embodiment of the medical isolation device 200 shown in FIGS. 15 and 16, the body 208 includes a collapsible frame 210 and a sheet material 212 overmolded onto the frame 210. The frame 210 includes first, second, and third arcuate shaped members 210a, 210b, 210c that meet at outer ends thereof. During use, each first outer end 210a-1, 210b-1, 210c-1 is attached to a first mount 214a, and each second outer end 210a-2, 210b-2, 210c-2 is attached to a second mount 214b. The mounds 214 secure the first, second, and third members 210 in position relative to one another, expanding the sheet material 212 into a dome-like shape. As with the medical isolation device 100 described above, the members 210a, 210b, 210c may have shapes other than arcuate, such as a rectangular or polygonal shape to define a rectangular body 208, or other desired shape.

During use, the first, second, and third arcuate shaped members 210a, 210b, 210c are positioned within the mounts 214 to form an expanded, operative position shown in FIGS. 15 and 16. The outer ends 210a-1, 210a-2 of the first member 210a are secured to the first and second mounts 214a, 214b, respectively, and the first member 210a rests on the flat surface during use. The mounts 214a include openings 216, 218 for receiving the second and third members 210b, 210c. The openings 216, 218 may be fully enclosed within the mount 214, as shown in FIG. 15. Alternatively, the openings 216, 218 may be partially exposed along an inner surface of the mount 214, as shown in FIG. 16.

More specifically, the outer ends 210b-1, 210b-2 of the second member 210b are positioned within openings 216a, 216b in the first and second mounts 214a, 214b, respectively. The openings 216a, 216b are positioned on the mounts 214a, 214b so that the second member 208b forms an acute angle with the first member 208a. The outer ends 210c-1, 210c-2 of the third member 210c are positioned within openings 218a, 218b in the first and second mounts 214a, 214b, respectively. The openings 218a, 218b are positioned on the mounts 214a, 214b so that the third member 208c forms an obtuse angle with the first member 208a. In other embodiments, the second and third members 210b, 210c may form other angles with the first member 210a. In still other embodiments, the frame 208 may include additional or fewer members 210.

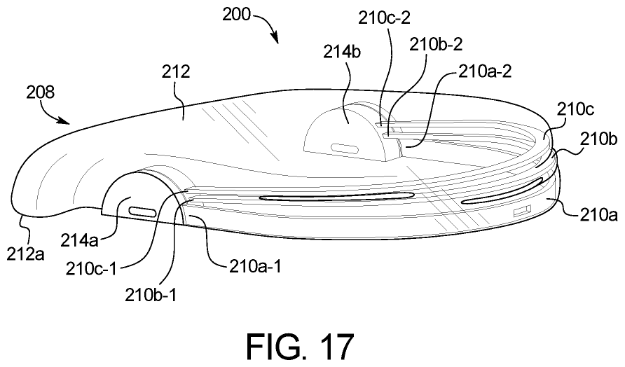

The sheet material 212 comprises a flexible material that folds upon itself into a collapsed position as shown in FIG. 17, but extends between the members 210 in the operative position shown in FIGS. 15 and 16 to form a sufficiently rigid structure that support portholes, porthole covers, and oxygen and suction ports and withstands negative pressure within the medical isolation device during use. Each mounts 214a, 214b includes a narrow slit 220a, 220b, respectively, that receives an edge 212a of the sheet material 212.

When the medical isolation device 200 is not in use, the body 208 is moved into the collapsed position shown in FIG. 17. Any porthole covers are first removed from the body 208. The outer ends 210b-1, 210b-2, 210c-1, 210c-2 of the second and third members 210b, 210c are then withdrawn from their respective openings 216, 218 in the respective mounts 214, and moved toward the first member 210a.

In alternative embodiments, the first, second, and third members 210a, 210b, 210c may include holes at the outer ends 210a-1, 210a-2, 210b-1, 210b-2, 210c-1, 210c-2. A stainless steel cotter style pin extends through the holes and includes a locking feature to maintain the first, second, and third members 210a, 210b, 210c in position relative to one another. Use of the pin maintains the frame 210 in the operative position, and withdrawal of the pin allows the members 210a, 210b, 210c and sheet material 212 to collapse into the collapsed position.

The sheet material is overmolded onto the frame 210 to form the body 208 of the medical isolation device 200. The first, second, and third members 210a, 210b, 210c may be made of aluminum, steel, or any other suitably rigid material. The sheet material within which the frame 210 is enveloped may be a silicone or other suitable material. As with the medical isolation device 100, the components of the body 208 are transparent, and allow for autoclaving and sterilizing as noted above.

Similar to the medical isolation device 100, the sheet of the body 208 includes one or more portholes, porthole covers, an oxygen port, and a suction port that are used to create negative pressure as described above. The collapsible body 208 of the medical isolation device 200 enables the device 200 to fold into a compact shape for easy storage and transport. The medical isolation device 200 may also include straps as noted above that extend below the operating table.

In still further embodiments, the medical device 100, 200 may include a body 108, 208 and drape 110 shaped and dimensioned to encompass the full body of the patient 104. In this embodiment, the body 108, 208 and/or the drape 110 may have a greater length.

It should be noted that various changes and modifications to the presently preferred embodiments described herein will be apparent to those skilled in the art. Such changes and modifications may be made without departing from the spirit and scope of the present invention and without diminishing its attendant advantages.

1. A medical isolation device comprising:
a body including an inner surface and an outer surface, the body also including a first side portion, a second side portion, a back side portion, and a top side portion, wherein a front side edge is formed along a first edge of the first side portion, an edge of the top side portion, and a first edge of the second side portion, wherein a base edge is formed along a second edge of the first side portion, an edge of the back side portion, and a second edge of the second side portion;
an oxygen port providing passage between the inner surface and the outer surface of the body;
a suction port providing passage between the inner surface and the outer surface of the body;
a porthole providing passage between the inner surface and the outer surface of the body,
a porthole cover positioned within the porthole, the porthole cover including an outer edge and an inner edge, the inner edge defining a throughway in the porthole cover, wherein, when no force is applied to the porthole cover, the cover is biased toward a folded position such that the inner edge comes together to close the throughway, and wherein, when force is applied to the porthole cover, the cover unfolds such that the inner edge is drawn apart to open the throughway; and
a drape attached to the body.

2. The medical isolation device of claim 1, wherein the base edge has an arcuate shape extending between first and second base edge endpoints, wherein the side edge has an arcuate shape extending between first and second side edge endpoints, and wherein the first and second base edge endpoints and the first and second side edge endpoints coincide.

3. The medical isolation device of claim 2, wherein the body has a dome shape extending between the base edge and the side edge, and wherein each of the base edge and the side edge has a U-shape.

4. The medical isolation device of claim 1, wherein the outer edge of the porthole cover includes a U-shaped groove for mating with a porthole edge of the porthole.

5. The medical isolation device of claim 1, further comprising a plurality of portholes and a plurality of porthole covers.

6. The medical isolation device of claim 1, wherein the drape has an attachment edge having a shape that corresponds with a shape of the front side edge of the body.

7. The medical isolation device of claim 6, wherein the attachment edge of the drape includes a U-shaped groove for mating with the front side edge of the body.

8. The medical isolation device of claim 6, wherein the attachment edge of the drape includes a plurality of pegs for mating with a plurality of openings on the front side edge of the body.

9. The medical isolation device of claim 6, wherein the drape includes a free edge opposite of the attachment edge.

10. The medical isolation device of claim 1, further comprising a pressure indicator.

11. The medical isolation device of claim 1, further comprising a filter attachment connected to the suction port.

12. The medical isolation device of claim 1, wherein the body includes a collapsible frame.

13. A method of using an intubation isolation device by a practitioner on a patient positioned on an operating bed, the method comprising:
providing a medical isolation device comprising:
a body including an inner surface and an outer surface, the body also including a first side portion, a second side portion, a back side portion, and a top side portion, wherein a front side edge is formed along a first edge of the first side portion, an edge of the top side portion, and a first edge of the second side portion, wherein a base edge is formed along a second edge of the first side portion, an edge of the back side portion, and a second edge of the second side portion;
an oxygen port providing passage between the inner surface and the outer surface of the body;
a suction port providing passage between the inner surface and the outer surface of the body;
a porthole providing passage between the inner surface and the outer surface of the body,
a porthole cover that inserts into the porthole, the porthole cover including an outer edge and an inner edge, the inner edge defining a throughway in the porthole cover, wherein, when no pressure is applied to the porthole cover, the cover is biased toward a folded position such that the inner edge comes together to close the throughway, and wherein, when pressure is applied to the porthole cover, the cover unfolds such that the inner edge is drawn apart to open the throughway; and
a drape attached to the body.
positioning the medical isolation device atop the operating bed such that a head of the patient is positioned within the body of the medical isolation device; and
attaching the drape to the side edge of the body such that a free edge of the drape rests atop a body of the patient;
connecting a suction tubing to the suction port on the medical isolation device, wherein the suction tubing is connected to a suction device;
connecting an oxygen tubing to the oxygen port on the medical isolation device, wherein the oxygen tubing is connected to an oxygen source; and
operating the suction device and the oxygen source to create airflow through the medical isolation device and maintain a negative pressure.

14. The method of claim 13, further comprising the steps of:
inserting, through the porthole cover, an arm of the practitioner, wherein the negative pressure within the medical isolation device is maintained while the arm extends through the porthole.

15. The method of claim 13, further comprising the steps of:
attaching an attachment edge of the drape to the front side edge of the body such that the free edge of the drape opposite of the attachment edge rests atop a body of the patient.

16. The method of claim 13, further comprising the steps of:
attaching a filter attachment to the suction tubing between the suction port and the suction device.
